(19) United States
(12) Patent Application Publication
(10) Pub. No.: US 2023/0420098 A1
(43) Pub. Date: Dec. 28, 2023

(54) SYSTEMS AND METHODS FOR QUANTIFYING PATIENT IMPROVEMENT THROUGH ARTIFICIAL INTELLIGENCE

(71) Applicants: Rehabilitation Institute of Chicago d/b/a Shirley Ryan AbilityLab, Chicago, IL (US); Northwestern University, Evanston, IL (US)

(72) Inventors: Stephen A. Antos, Chicago, IL (US); Konrad P. Kording, Philadelphia, PA (US); Vivek Sagar, Chicago, IL (US)

(21) Appl. No.: 18/250,140

(22) PCT Filed: Jan. 31, 2022
(86) PCT No.: PCT/US2022/014605

§ 371 Date: Apr. 21, 2023

Related U.S. Application Data
(60) Provisional application No. 63/143,543, filed on Jan. 29, 2021.

(51) Int. Cl. G16H 20/00; G16H 50/30

(52) U.S. Cl. CPC G16H 20/00; G16H 50/30

(57) ABSTRACT

Examples of a system and methods for quantifying patient improvement via artificial intelligence are disclosed. In general, via at least one processing element, a machine learning model such as a Siamese neural network is trained in view of a cost function to learn on average a maximum difference in outcomes between a patient at different points in time. Given the architecture of the neural network, a plurality of outcome measures generated for a given point in time can be condensed into a single score.

### CROSS REFERENCE TO RELATED APPLICATIONS

This is a PCT application that claims benefit to U.S. provisional application Ser. No. 63/143,543 filed on 29 Jan. 2021 entitled SYSTEMS AND METHODS FOR GENERATING OUTCOME MEASURES which is incorporated by reference in its entirety.

### FIELD

Aspects of the present disclosure relate generally to computer-implemented rehabilitative systems; and in particular to a system and associated methods for quantifying patient improvement using artificial intelligence such as neural networks.

### BACKGROUND

Rehabilitation outcome measures provide clinically useful data to demonstrate patient improvement, guide treatments, and justify services. Currently, there are hundreds of rehabilitation outcome measures that clinicians can use. Outcome measures can be self-reported or performance based, and address different domains such as mobility, activities of daily living, or cognition. Some outcome measures are targeted for specific diagnoses, whereas others are meant to be applied more broadly. The wide array of available outcome measures provides clinicians with an extensive library of tools to assess patient ability. However, it is technically challenging to identify relevant trends and observations in rehabilitative change among many different outcome measures.

It is with these observations in mind, among others, that various aspects of the present disclosure were conceived and developed.

### SUMMARY

The following presents a simplified summary of various aspects described herein. This summary is not an extensive overview and is not intended to identify key or critical elements or to delineate the scope of the claims. The following summary merely presents some concepts in a simplified form as an introductory prelude to the more detailed description provided below. Corresponding apparatus, systems, and computer-readable media are also within the scope of the disclosure.

Outcome measures are becoming increasingly important as healthcare payors move away from fee-for-service reimbursement to value-based care models. Value is often represented as a conceptual equation: patient improvement divided by the cost of care. Cost is relatively easy to determine through patient billing and payor reimbursement; however, quantifying improvement is much more difficult because there can be multiple outcome measures, and the measures chosen can vary from patient to patient. A standard set of outcome measures is one potential solution. However, developing a standard battery of assessments across all patients is challenging because some measures may be unsafe, insensitive, or invalid for some patient populations. Furthermore, performing unnecessary or inappropriate outcome measures wastes resources such as computational load, and decreases the efficiency of care. Even if a standardized set of outcome measures existed for all patients, clinicians or payers would still be left with the task of quantifying overall improvement from multiple measurements. Hundreds, if not thousands, of outcome measures and biomarkers can be tracked over time to evaluate a patient's response to medical services. However, interpreting hundreds or thousands of measurements simultaneously is intractable. A universal method to combine such measurements into lessor overall numbers and/or a single number representing improvement would enable the estimation of value.

Examples of a novel concept herein are derived from a challenge or problem associated with rehabilitative systems in that the patient's improvement or change in ability, is a latent construct that cannot be measured, and that data analysis of voluminous amounts of outcome measures is inefficient and does not produce viable results. It is argued that clinicians and payers can, at best, infer a patient's improvement using observable measurements (i.e., outcome measures). As a technical solution responsive to the foregoing challenges of dealing with outcome measures, examples of the present novel concept utilize a practical application of machine learning to quantify or estimate improvement that incorporates an assumption that patients are admitted to inpatient rehabilitation at a given ability level and leave inpatient rehabilitation with a new ability level. On average, a patient's ability improves from admission to discharge because inpatient rehabilitation is the best available intervention for that patient. Furthermore, skilled clinicians choose outcome measures that will provide the most relevant data to infer a patient's ability.

In one specific example, the present inventive concept can take the form of a computer-implemented method, comprising the steps of accessing, by a computing device, a first dataset of input data for one or more outcome measures derived from a patient at a first point in time of rehabilitation; accessing, by the computing device, a second dataset of the input data for the one or more outcome measures derived from the patient at a second point in time of the rehabilitation; and generating, by the computing device applying the first dataset and the second dataset as inputs to a machine learning model, an output including a machine learning score that infers improvement of the patient from the first point in time to the second point in time, the machine learning model trained to map the inputs to the output to minimize a cost function defined by the machine learning model and maximize the dissimilarity of the patient (but may be trained using a plurality of patients) between the first point in time and the second point in time. The machine learning model may be a Siamese neural network trained that minimizes the cost function based on training data defining outcome measures fed to the machine learning model during training.

In another example, the present inventive concept can take the form of a system comprising a memory storing instructions, and a processor in operable communication with the memory that executes the instructions to: train a Siamese neural network to learn a mapping from inputs defining a plurality of outcome measures to its output, a single intermediate score, to maximize its cost function. The Siamese neural network includes an input layer including a node for each outcome measure, and an output layer including a sole node that provides the single intermediate score.

In yet another example, the present inventive concept can take the form of a tangible, non-transitory, computer-readable medium having instructions encoded thereon, the instructions, when executed by a processor, being operable to: generate a machine learning score reflecting a total difference in a patient between a first point in time and a second point in time by feeding a first set of outcome measures to a neural network and a second set of outcome measures to the neural network, the neural network trained to minimize a cost function associated with the neural network and maximize the dissimilarity between the patient between the first point in time and the second point in time.

These examples and features, along with many others, are discussed in greater detail below.

### BRIEF DESCRIPTION OF THE DRAWINGS

FIG. 1 is a simplified block diagram of an example computer-implemented system for quantifying patient improvement via artificial intelligence as described herein.

FIG. 2A is an example process associated with the inventive concept for quantifying patient improvement via artificial intelligence as described herein.

FIG. 2B is another example process for quantifying patient improvement via artificial intelligence as described herein.

FIG. 3A is an illustration of general data flow for implementing the concepts described herein.

FIG. 3B is an illustration of patient data associated with rehabilitation including a plurality of outcome measures of a patient.

FIG. 4A is an illustration of a machine learning phase performed by at least one processing element which may be implemented for quantifying patient improvement using at least one neural network.

FIG. 4B is an illustration of a testing and/or implementation phase performed by at least one processing element to test and/or implement the neural network from FIG. 4A as trained.

FIG. 4C is another illustration of a testing and/or implementation phase performed by at least one processing element to test and/or implement the neural network from FIG. 4A as trained.

FIG. 5A is an illustration of patient data at admission and at discharge with various outcome measures entries being missing or devoid of data.

FIG. 5B is an illustration of the admission patient data as missing values are being computed as described herein.

FIG. 5C is an illustration of the discharge patient data as missing values are being computed as described herein.

FIG. 5D is an illustration of the patient data of FIG. 5A with computed values replacing the missing data, as described herein.

FIG. 6A is an illustration of an exemplary technical operating environment for implementing functionality described herein.

FIG. 6B is a simplified block diagram of an exemplary computing device that may be implemented to execute functionality described herein.

Corresponding reference characters indicate corresponding elements among the view of the drawings. The headings used in the figures do not limit the scope of the claims.

### DETAILED DESCRIPTION

Described herein are examples of computer-implemented systems and methods that relate to quantification of patient improvement using artificial intelligence. In various instances, machine learning can be implemented by one or more processing elements to train a machine learning model such as a neural network to take any set of numeric outcome measures and biomarkers before and after treatment (and/or at two or more predetermined points in time) and generate a distribution of scores reflecting a computed difference in the patient. More specifically, a first set of outcome measures associated with a first point in time may be fed to the trained machine learning model to compute a first intermediate score, and a second set of outcome measures associated with a second point in time may be fed to the trained machine learning model to compute a second intermediate score; the difference between the second intermediate score and the first intermediate score defining a machine learning (ML) score reflecting a total difference in the patient between the first point in time and the second point in time. While there are infinite ways to combine outcome measures into a single intermediate score, it is the way they are combined according to the novel examples described herein (e.g., trained neural networks) which dictate the properties of the intermediate scores and make it meaningful.

On average, patients improve in response to medical treatments because clinicians typically choose the most effective intervention available. For example, in the domain of acute inpatient rehabilitation we can assume that, on average, acute inpatient rehabilitation has a maximal effect on improvement between admission and discharge. The machine learning model can use this assumption as its objective or cost function and compress outcome measures into a single score reflecting the dissimilarity between a patient at admission and discharge. The ML score reflecting dissimilarity (e.g., between outcome measures) represents a difference and/or the change in ability (i.e., improvement) between two points in time. In some examples, the machine learning model can be trained to find the maximum effect of the treatment for that population, based on the assumption that the intervention and outcome measures chosen are best for the patient. Once trained, the machine learning model can generate improvement scores for new patients and can be used to identify potential treatments for patients. For example, potential treatments can be analyzed based on the outcome scores in view of the past treatment given, and treatments can be recommended for a patient based on what has been successful in the past. Such potential treatments can then be administered to the patients or new patients.

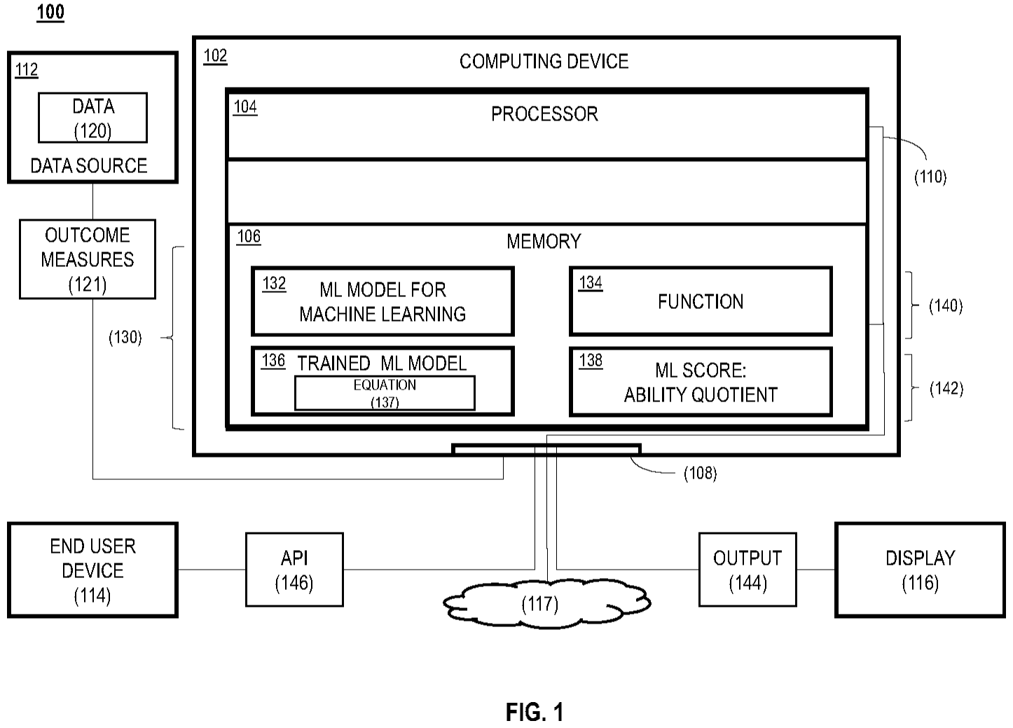

Referring to FIG. 1, an example of the novel concept described includes a (computer-implemented) system 100 for quantification of patient improvement using machine learning or other forms of artificial intelligence. The system 100 comprises any number of computing devices or processing elements. In general, the system 100 leverages artificial intelligence to implement predictive machine learning methods for quantifying patient improvement. While the present inventive concept is described primarily as an implementation of the system, it should be appreciated that the inventive concept may also take the form of tangible, non-transitory, computer-readable media having instructions encoded thereon and executable by a processor, and any number of methods related to embodiments of the system described herein.

The system 100 includes (at least one of) a computing device 102 including a processor 104, a memory 106 of the computing device 102 (or separately implemented), a network interface (or multiple network interfaces) 108, and a bus 110 (or wireless medium) for interconnecting the aforementioned components. The network interface 108 includes the mechanical, electrical, and signaling circuitry for communicating data over links (e.g., wires or wireless links) within a network (e.g., the Internet). The network interface 108 may be configured to transmit and/or receive data using a variety of different communication protocols, as will be understood by those skilled in the art. As further shown, the computing device 102 may be in operable communication with at least one data source 112, at least one of an end-user device 114 such as a laptop or general purpose computing device, and a display 116. The system may further include a cloud 117 or cloud-based platform (e.g., Amazon® Web Services) for implementing any of the training and implementation of machine learning models described herein.

In general, via the network interface 108 or otherwise, the computing device 102 is adapted to access data 120 including outcome measures 121 from one or more of the data sources 112. The data 120 accessed may generally define or be organized into datasets or any predetermined data structures which may be aggregated or accessed by the computing device 102 and may be organized within a database stored in the memory 106 or otherwise stored. The data 120 may include without limitation training datasets including sets of the outcome measures 121 for patients over time where such training datasets are historical or otherwise suitable for training a machine learning model, and/or distributions of outcomes measures 121 over time for a patient where analysis of the outcome measures 121 for the patient has not been conducted (i.e., live or non-analyzed data).

In some examples, the processor 104 of the computing device 102 is operable to execute any number of instructions 130 within the memory 106 to perform operations associated with training a machine learning model 132 and/or conducting machine learning, implementing a cost function 134 that assists with the machine learning, testing or otherwise implementing a trained machine learning (ML) model 136 defining at least one equation 137, and generating a machine learning score 138 by implementing the trained ML model 136 as described herein. In general, the system 100 is configured to compute the trained ML model 136 (including the equation 137 with various configured weights, biases, and parameters) by applying machine learning 132 in view of the cost function 134 to training datasets defined by the data 120 (during a training phase 140), so that the trained ML model 136 when executed by the processor 104 in view of new outcome measures 121 outputs an ML score 138 indicating a difference in a patient over time (during a testing and/or implementation phase 142) based on the new outcome measures 121. Aspects may be rendered via an output 144 to the display 116 (e.g., a graph or report illustrating patient improvement by the computed ML score 138 over time), and aspects may be accessed by the end user device 114 via one or more of an application programming interface (API) 146 or otherwise accessed.

The instructions 130 may include any number of components or modules executed by the processor 104 or otherwise implemented. Accordingly, in some embodiments, one or more of the instructions 130 may be implemented as code and/or machine-executable instructions executable by the processor 104 that may represent one or more of a procedure, a function, a subprogram, a program, a routine, a subroutine, a module, an object, a software package, a class, or any combination of instructions, data structures, or program statements, and the like. In other words, one or more of the instructions 130 described herein may be implemented by hardware, software, firmware, middleware, microcode, hardware description languages, or any combination thereof. When implemented in software, firmware, middleware or microcode, the program code or code segments to perform the necessary tasks (e.g., a computer-program product) may be stored in a computer-readable or machine-readable medium (e.g., the memory 106), and the processor 104 performs the tasks defined by the code.

Exemplary Processes

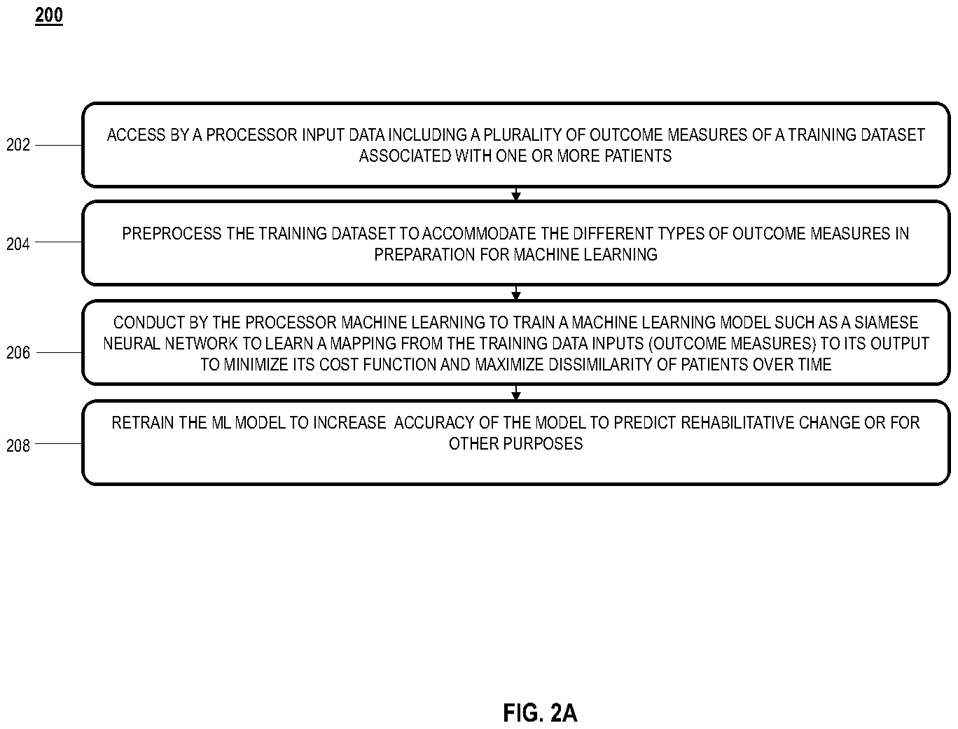

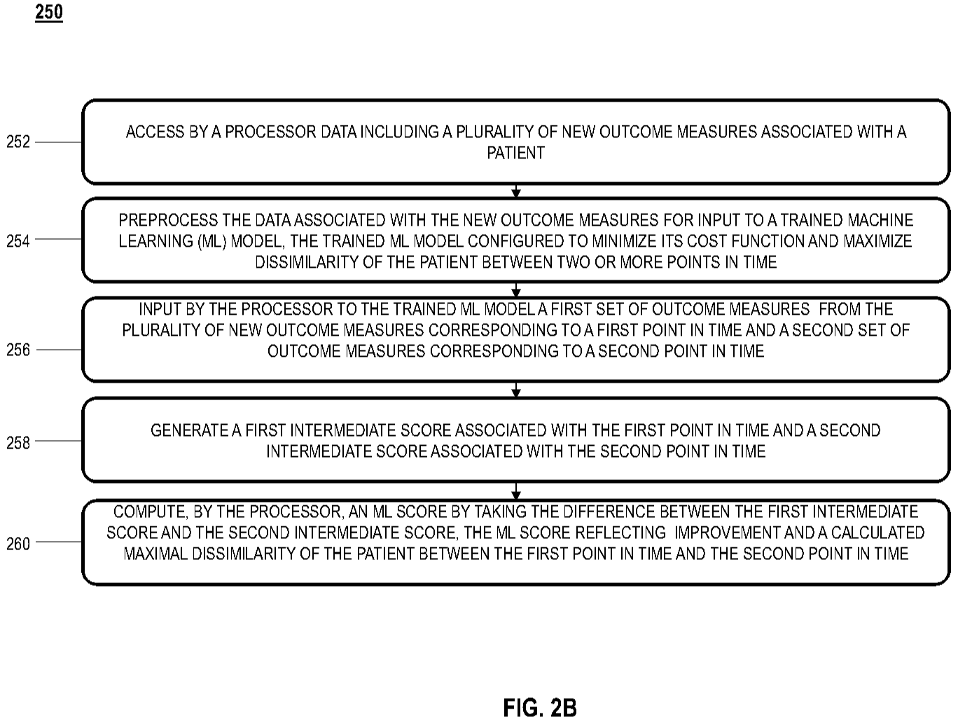

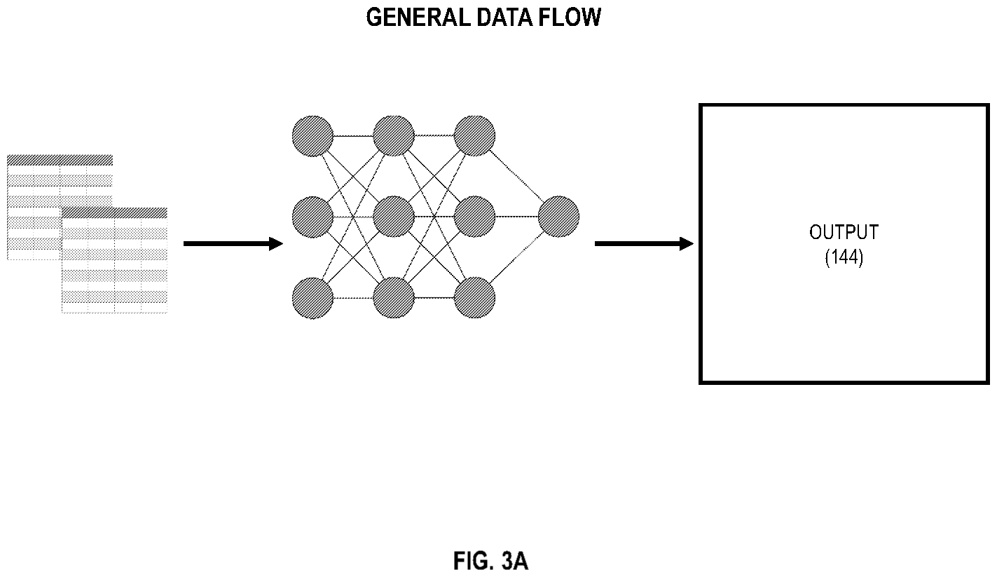

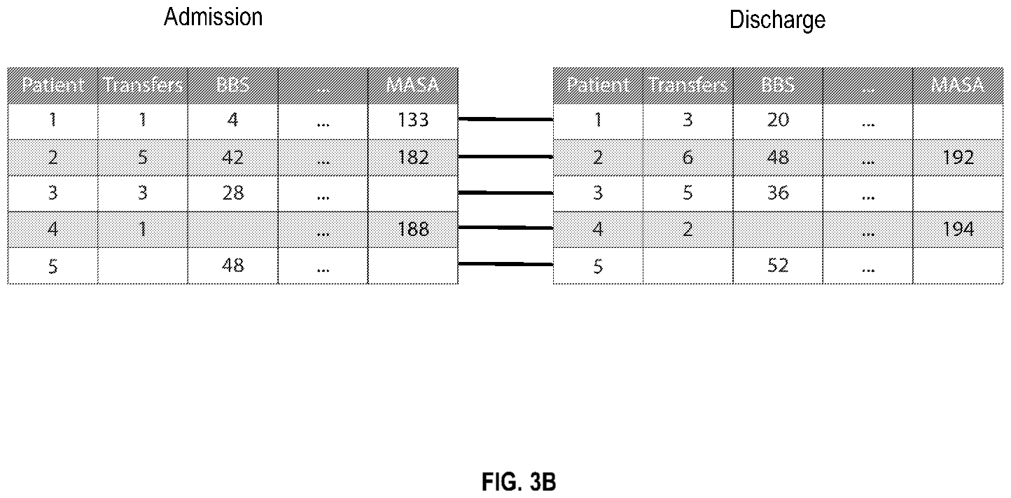

Referring to FIGS. 2A-2B and FIGS. 3A-3B, via one or more processing elements such as the processor 104, one or more machine learning models may be trained, tested, and implemented to quantify patient improvement using artificial intelligence such as neural networks. In general, outcome measures 121, examples in FIG. 3B, are fed to the machine learning model as trained to generate any number of outputs 144 viewable via the display 116 or otherwise. Any number of ML models may be trained and implemented, and ML models may be trained for specific groups of outcome measures 121 or any predetermined rehabilitative procedures.

To illustrate the training phase 140 of FIG. 2A, an exemplary computer-implemented process 200 may be performed by the processor 104 or other processing element to train a machine learning model 132 such as a neural network. Any number or type of the outcome measures 121 can be used to train the ML model 132. By training many or all known/relevant outcome measures across an entire population or predetermined demographic, the machine learning model 132 learns which measurements are most reliable and show the greatest difference for the intervention (i.e., inpatient rehabilitation).

As shown in block 202 of the trained process 200, the processor 104 accesses from the data 120 a plurality of outcome measures 121 as a training dataset. Outcome measures 121 include functional independence measures (FIMs), or any measure suitable for assessing rehabilitative change of a patient. Accordingly, outcome measures can include a variety of ordinal, interval, and/or ratio data types. For example, outcome measures can include everyday activities, such as bed chair transfer, locomotion (walk), locomotion (wheelchair), locomotion (stairs), eating, grooming, bathing, dressing (upper), dressing (lower), toileting, toilet transfer, tub shower transfer, comprehension, expression, social interaction, problem solving, memory, bladder management, bowel management, and/or the like. Outcome measures can also include performance on a variety of assessment tests, such as action research arm test, berg balance scale, box and blocks (r & l arms), coma recovery scale, functional assessment of verbal reasoning, function in sitting test, five times sit to stand, functional oral intake scale, functional gait assessment, head control, Kessler Foundation neglect assessment, Mann assessment of swallowing, orientation o-log, pressure relief, six minute push test, six minute walk test, ten meter walk test, three word delayed recall, walking index for spinal cord injury, and the like.

Moreover, non-numeric outcome measures 121 can be converted to numerical values and applied. As a non-limiting example, images of some portion of a patient's body can be broken into features and numerical values to assess some rehabilitative change of the patient. In this manner, any value informative as to a possible change of the patient over time can be applied as an “outcome measure” (121).

Referring to block 204, by the processor 104, the training dataset of outcome measures 121 is preprocessed, standardized, and/or normalized in preparation for machine learning by the machine learning model 132. In some examples, values of the training dataset are rescaled for each outcome measure to a range of [0,1] using the minimum and maximum values for each outcome measure. Any number or type of preprocessing procedures may be executed. For example, the outcome measures 121 of the training dataset may be formatted, preprocessing may include feature extraction, data may be filtered, and the like. In addition, the step of block 204 can include forward filling. In addition, a number of columns of the data of the training dataset can be doubled and a “mask” can be created to address possible missing values of the outcome measures 121. It should also be understood that acquisition of the outcome measures 121 may include acquisition of both the training dataset described and a testing dataset. In other words, preprocessing may include dividing data between the training dataset and a testing dataset referenced below.

In general, the machine learning model 132 is given two input training datasets (or subsets of a training dataset). In some examples a dataset is provided for each point in time (e.g., an exemplary case would include an admission dataset and a discharge dataset). Features may be normalized to fit the range [0,1]. Features could also be standardized or transformed as needed pending the application of the neural network. Feature matrices can include any traditional approaches for machine learning applications.

An example of outcome measures 121 acquired during preprocessing is shown in FIG. 3B. In this example, two sets of outcome measures 121 are populated within respective tables as shown indicating patient outcome measures at admission and at discharge. Each table row represents a patient, and each table column represents an outcome measure. In some examples, the tables are linked: the same patient gets the same row in each table. As further illustrated in FIG. 3B, values may be missing from the data acquisition, and the present disclosure includes the following novel approach to addressing such missing data.

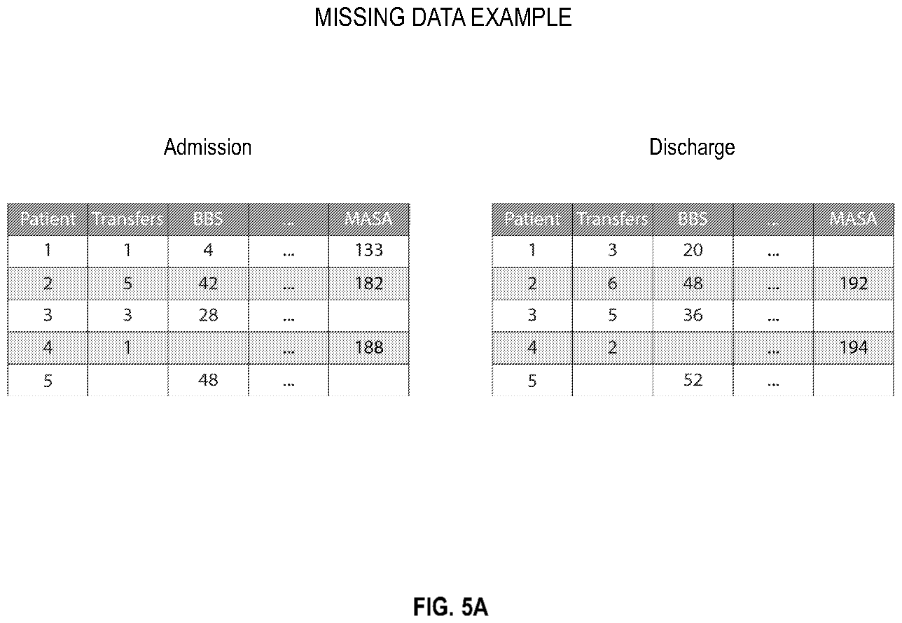

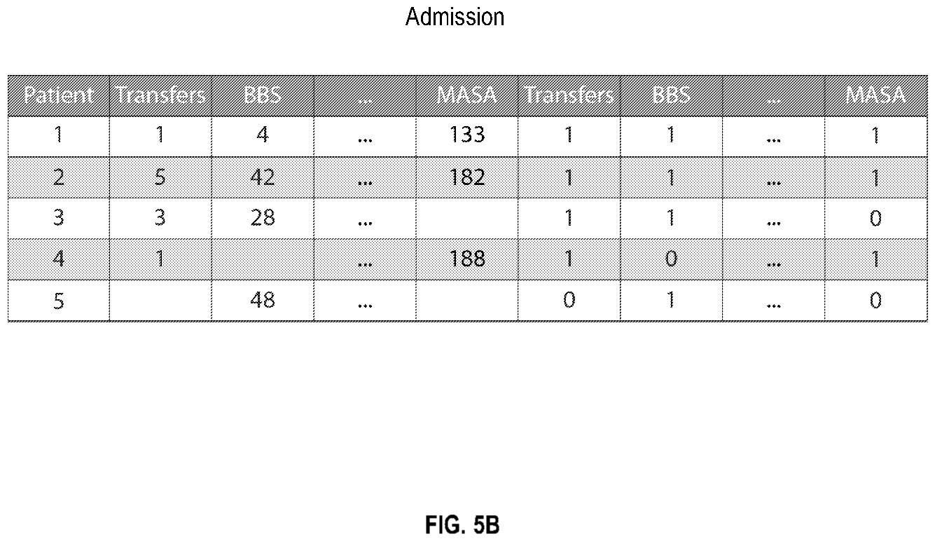

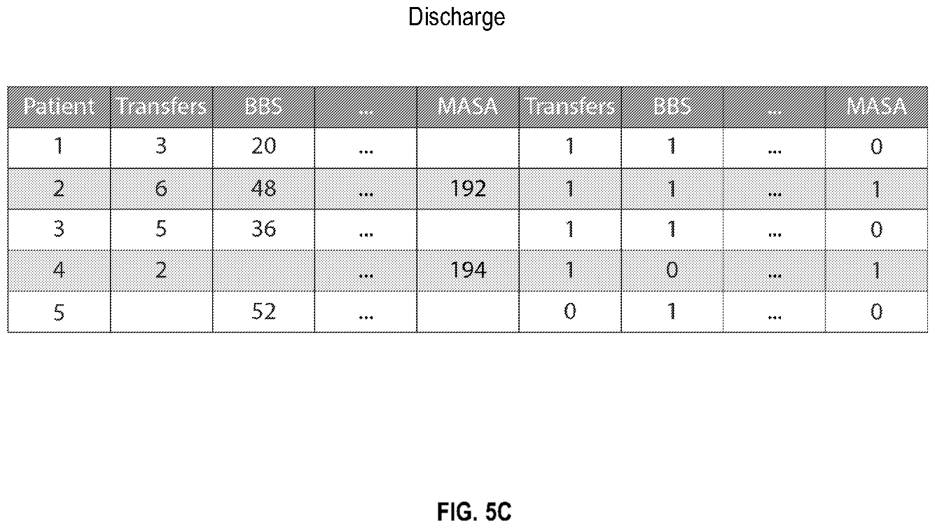

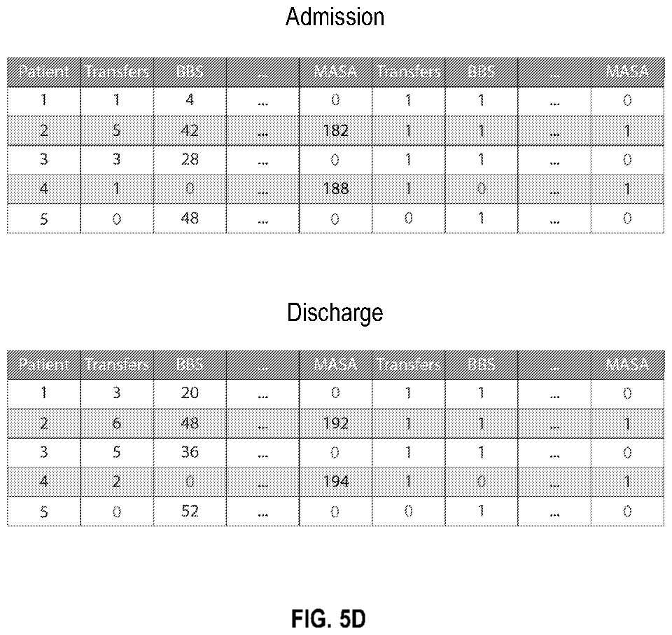

Missing Data (FIGS. 5A-5D): For each feature in a feature matrix, an additional column can be appended to the input training datasets. These appended columns serve as a “mask” to indicate whether an outcome was measured or not measured (i.e. missing). For each patient, a value can be set to 1 if a measurement was present for a specific outcome measure. In contrast, the value is set to 0 if missing. In some examples, the neural network requires an outcome measure to be present in both input datasets relating to different points in time (i.e. admission and discharge). If only one measurement is present, then we set both the measurements (for admission and discharge datasets) and both “mask” columns to 0. This removes any information about the outcome measure for patients across the two time points. We perform this step because a “0” on a specific outcome measure may have meaning that is different from a “0” if nothing was measured at all. The neural network machine learning model 132 learns to find differences, and to use an outcome measure it must be present at both points in time for the machine learning model 132 to learn.

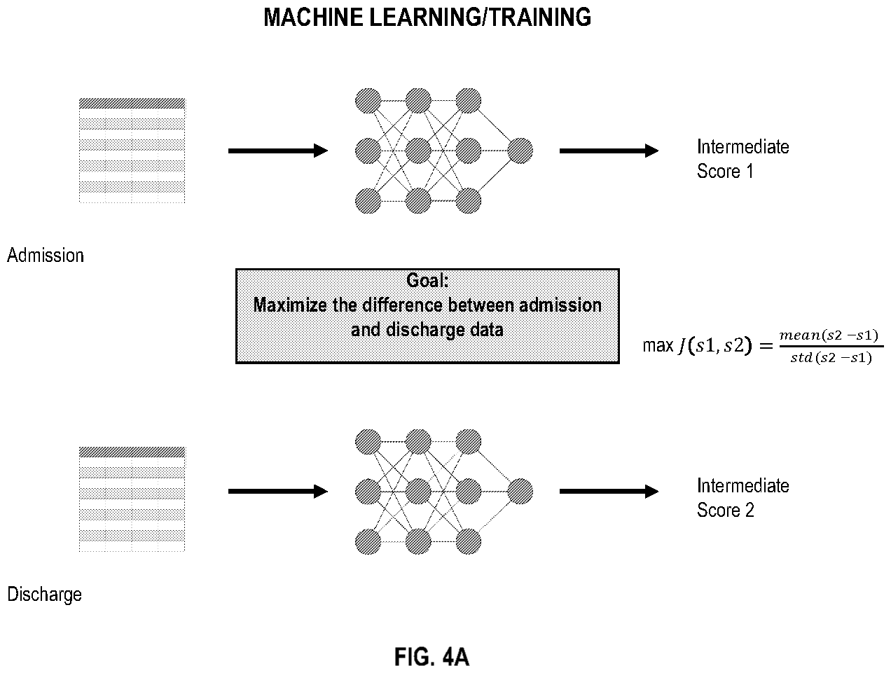

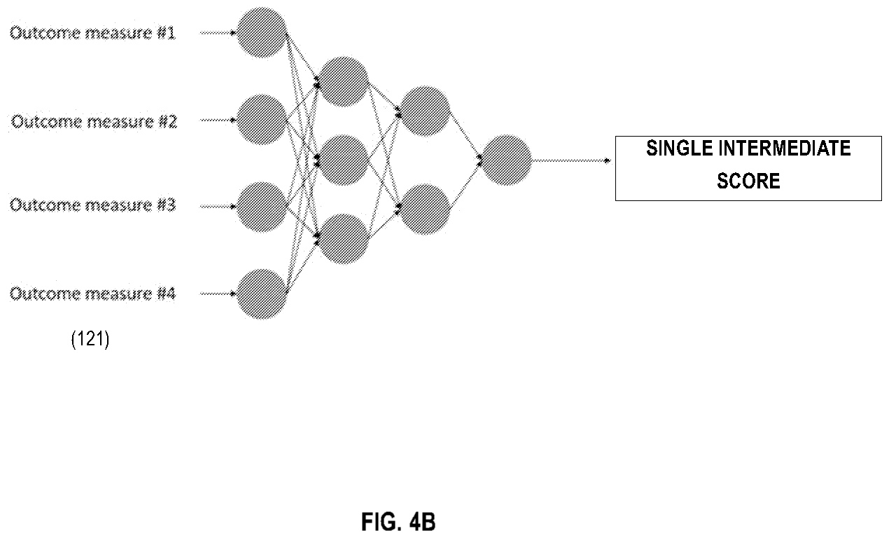

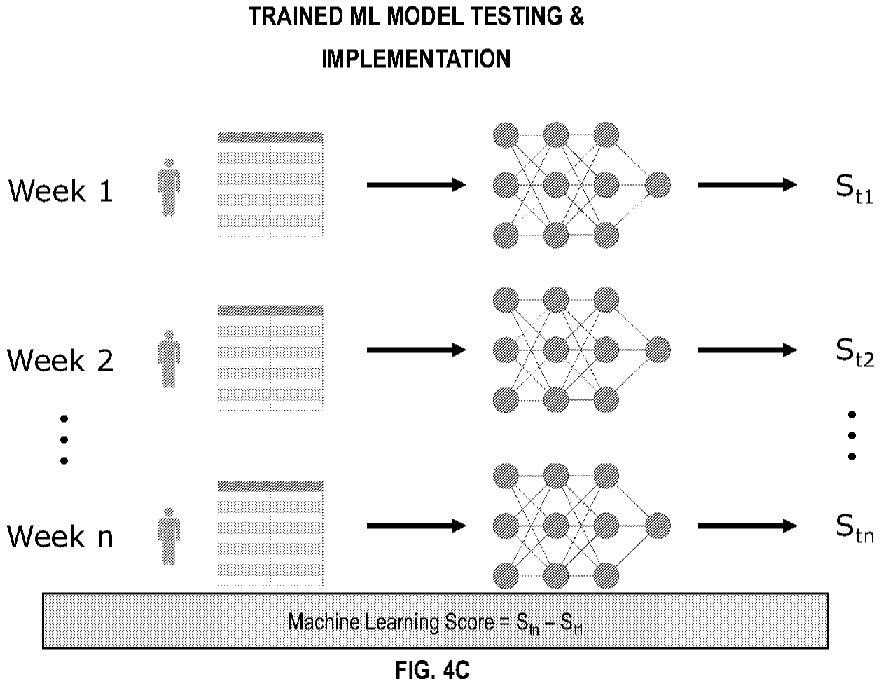

Referring to block 206 and FIGS. 4A-4B, the processor 104 accesses the general machine learning (132) model such as a neural network for machine learning in view of the training dataset of outcome measures 121 of block 204 (i.e., the training dataset of the outcome measures 121 is fed to the neural network). In some examples, the machine learning model (132) is implemented as a variation of a Siamese neural network (SNN). Siamese Neural Networks (SNNs) are a type of machine learning model determined by the present inventive concept to be particularly suitable for analyzing numerous outcomes measures of a patient or a plurality of patients. Typically used for one-shot image classification, SNNs consist of two “twin” neural networks that have identical architectures and weights. Traditionally, thousands of image pairs are fed into the network, and the network learns what makes each image pair similar or dissimilar using a contrastive objective function. Once trained, the network can take a pair of new images and produce a similarity score to determine whether the images belong to the same class.

In the present example, an architecture is employed where the Siamese neural network is combined with the cost function 134. In other words, the cost function 134, described further herein, can be a contrastive objective/cost function that allows the underlying Siamese neural network to learn about the outcome measures 121 data in a unique way as the training dataset is fed to the neural network. Instead of the neural network learning to contrast images based on their pixels, the neural network learns to contrast patients based on their outcome measures 121. Instead of learning to generate a similarity score between two images and using it for classification, the neural network learns to generate a patient's dissimilarity (ML) score 138 between two time points and the dissimilarity itself provides a measure of improvement. The cost function 134 determines the properties and final distribution of dissimilarity scores (i.e. improvement).

While there are infinite ways to combine outcome measures 121 into a single score, it is the way they are combined under the present novel disclosure which dictate the properties of a final score and make it meaningful. The approach described herein is based on that presumption that on average, patients improve in response to medical treatments because clinicians choose the most effective intervention available. For example, in the domain of acute inpatient rehabilitation we can assume that, on average, acute inpatient rehabilitation has a maximal effect on improvement between admission and discharge. A Siamese Neural Network uses this assumption as its objective function (cost function 134) and compresses outcome measures into a single score reflecting the dissimilarity between a patient at admission and discharge. Because the inputs to the neural network are outcome measures meant to measure progress, it is proposed that the dissimilarity (ML) score 138 represents the change in ability (i.e. improvement) between two points in time.

Examples of the cost function 134 are provided below. The cost function 134 can be considered a cost, loss, and/or objective function. In general, the SNN learns a mapping from its inputs (outcome measures 121) to its output (ML score 138) to minimize its (cost) cost function 134 and maximize dissimilarity to estimate the effect of inpatient rehabilitation on patient ability. The SNN learns to detect differences in outcome measures of patients over time, reflected by the ML score 138.

Example 1 of cost function 134. A general implementation of the cost function 134 is as follows:

J min(s1,s2)=−mean(s2−s1)/std(s2−s1)

In this example, the cost function 134 assists the neural network to learn to maximize the difference between admission and discharge data. Admission data is represented as S1 and can include data associated with any first point in time, and discharge data is represented as S2 and can include any data associated with a point in after the first point in time.

Example 2 of cost function 134. In this example we refer to the change in patient status between two points in time (e.g., admission and discharge) as ability:

$${J}_{min} = \frac{- E [ Δ Ability ]}{\sqrt{E [ Δ {Ability}^{2} ] - {E [ Δ Ability ]}^{2}}} \qquad Eqn 2$$

In other words, the ML model 132 tries to learn to maximize the difference in patient ability between admission and discharge or two points in time.

The ML model 132 in some examples is a fully connected Siamese Multilayer Perceptron with two hidden layers: an input layer and an output layer. The input layer has one node for each outcome measure, and the output layer has one node that computes the final (difference) ML score 138. In some example implementations, a dropout rate of 25% for the input and hidden layers, and L2 (ridge regression) regularization for the weights (beta=0.0001) can be used, and also an Adam optimizer with a learning rate of 0.001. The number of hidden layers, number of nodes in the hidden layers, dropout rate, L2 regularization penalty, optimizer, and optimizer parameters can all be tuned or changed depending on the application of the ML model 132. For example, an exponential decay can be applied to the learning rate to ensure network convergence.

At block 206, during machine learning (training phase 140), where the machine learning model 132 is a Siamese neural network, the training dataset is fed through two networks that share the cost function 134. On each update, both networks are changed simultaneously with an identical update. This ensures that the networks remain identical throughout the training process. In some examples, a 50-50 train-test split can be used and the machine learning model 132 can be trained for 200 epochs. In practice, the number of epochs can vary or be tuned. We can also generate an ensemble of models, by bootstrapping the training set for the original population.

Processing the outcomes measures 121 during machine learning of the ML model 132 (or otherwise feeding the outcome measures to the ML model 132) can be described as “compressing” the outcomes measures 121, by going from many inputs (a plurality of outcome measures 121) to one output (intermediate score). The process can be visualized for demonstration purposes as a funnel, and nodes of the neural network can be expanded/increased; i.e., additional outcomes measures can be considered by the machine learning model 132. To illustrate, FIG. 4B illustrates outcome measures 121 associated with some point in time being fed to a neural network. Each arrow of FIG. 4B represents a computation that happens as outcome measures 121 are fed to the neural network during training and/or testing. These computations keep carrying forward until you reach a final number at the end of the network, or a “single intermediate score.” As such, the ML model 132 can essentially be considered to define a larger overall equation (collectively “equation 137” in FIG. 1) that takes all the outcome measures for a given point in time and computes a single number for that point in time.

During training, the equation 137 (comprising any number of equations and/or mathematical functions defined by the neural network) is learned. The cost function 134 informs the neural network how to “learn” what the equation 137 should be. The cost function 134 effectively asks the neural network to learn the equation 137 that, on average, finds the largest difference in patients between two points in time, e.g., admission and discharge (i.e. be as sensitive as possible to differences in outcomes).

More specifically, the neural network modifies its parameters (weights and biases) to minimize the cost function (134) based on the data provided (outcome measures 121). During training, the neural network is given input data, computes the output, and then calculates the current cost using the cost function 134. It uses a form of gradient descent and back-propagation to update its weights and biases in a way that improves its cost (i.e. learning). As indicated in block 208, during training, this cycle may be continued and/or repeated of giving the neural network inputs, computing outputs, calculating cost, and updating the network parameters. This process can be implemented hundreds if not thousands of times, so the neural network learns the best parameters for the equation 137 to minimize its cost function 134.

There are many hyperparameters associated with the equation 137 that can be modified and/or tuned during learning. For example, the choice of optimizer, the dropout, the regularization, the learning rate, the keep probability, the beta or regularization parameter, and the activation function, among other non-limiting examples. In addition, the ML model 132 can be modified as desired based on more or less outcomes measures data. For example, the nodes of the neural network example of the ML model 132 can be modified, and/or layers of the neural network can be increased or decreased. During training, epochs, defining the number of times the training dataset is passed through the ML model 132 during training can be predetermined. A number of different trained models can be generated during the training phase 140, referred to in the art as ensembles or the number of models desired. The initial training dataset can be broken down into smaller batches and sent one at a time through the ML model 132 to learn.

Referring to FIG. 2B illustrating an exemplary process 250 for a training/implementation phase 142, the trained ML model 136, representing the ML model 132 trained using the cost function 134, can be tested and/otherwise implemented to assess a patient's change over time. In general, once the ML model 132 is trained to form the trained ML model 136, new patient information from any two time points can be input into the trained ML model 136 or any ensemble of models similarly trained to obtain a distribution of difference scores to use or interpret. In this case, the higher (or more positive) the difference score, the greater the improvement the patient made during inpatient rehabilitation. If the difference score is negative, this means the patient regressed during inpatient rehabilitation.

Referring to blocks 252 and 254 (FIG. 2B), similar to blocks 202 and 204 of process 200 (and incorporating features thereof), a first plurality of outcome measures 121 associated with a first point in time (such as admission), and a second plurality of outcome measures associated with a second point in time (such as discharge) is accessed by the processor 104. Data associated with the first plurality of outcome measures 121 and the second plurality of outcome measures 121 can be preprocessed using any of the features described in block 204.

Referring to blocks 256, 258, and 260, once trained, new patient information from any two time points can be input into trained ML model 136 and/or any similarly trained ensemble of models to compute by the processor 104 a distribution of difference scores to use or interpret. The higher (or more positive) the difference score, the greater the improvement the patient made during inpatient rehabilitation. If the difference score is negative, this means the patient regressed during inpatient rehabilitation.

To illustrate, as shown in FIG. 4C, a first set of outcome scores associated with a first point in time (ₜ₁), illustrated as Week 1, can be fed to the trained ML model 136 to compute a first intermediate score, designated St₁. Similarly, a second set of outcome scores associated with a second point in time (ₜ₂), illustrated as Week 2, can be fed to the trained ML model 136 to compute a second intermediate score, designated St₂. The ML learning score 138, reflecting a total difference in the patient between the first point in time and the second point in time may further be computed as shown by taking the difference between St₂ and St₁. As indicated, the same comparative computations can be performed for subsequent weeks such as a third week or fourth week and the like.

FIGS. 5A-5D illustrate methods for addressing missing data, as indicated above. FIG. 5A illustrates a fictional example of how to handle missing data for five patients. Each patient has outcomes measured at admission and discharge. We show three potential outcomes that are measured for each patient in this figure: FIM Transfers, the Berg Balance Scale (BBS), and the Mann Assessment of Swallowing Ability (MASA). The ellipsis represents any other numerical outcomes measures for these patients. FIG. 5B looks at the admission data set and begins the masking procedure. We append a new column for each outcome measure. If an outcome measure is present in this scenario, the correspond patient and outcome measure column is marked as a 1 (present). If a patient's data is missing for an outcome measure, it is marked as a 0 (missing). FIG. 5C shows the same as FIG. 5B and also shows the same process for the discharge data. Referencing FIG. 5D, taking what we have in FIGS. 5B-5C, we finish “masking” the data. If a patient has an outcome measure at both admission and discharge, we do not change these entries. However, if an outcome is missing at either admission or discharge, we replace the outcome measure value (missing or present) with a 0 in both the admission and discharge datasets. We also set the value of the corresponding “mask” column to 0 in both the admission and discharge datasets.

An exemplary algorithmic description based on FIGS. 5A-5D is as follows:

### Example: Patient 1, MASA

FIG. 5A:
Masa column on admission: 133
Masa column on discharge: missing

FIG. 5B:
Masa column on admission: 133
Masked Masa column on admission: 1 (present)

FIG. 5C:
Masa column on discharge: missing
Masked Masa column on discharge: 0 (missing)

FIG. 5D:
Discharge data is missing masa, so we do the following
Set Masa column on admission=0
Set Masked Masa column on admission=0
Set Masa column on discharge=0
Set Masked Masa column on discharge=0
If we look at BBS for patient 1, the measures are retained because they are present at both time points, and the masked columns are set to 1.

### Administering Treatment

Using intelligence uncovered by training and application of the trained ML model 136, treatments can be tailored for patients according to one or more aspects of the disclosure. For example, patient data can be obtained. The patient data can describe any of a variety of attributes of a patient, such as conditions being experienced by the patient, the medical history of the patient, and the like. Current condition data can then be determined. The current condition data can indicate a patient's ability level for one or more activities. The current condition data can include both a patient's initial ability level and/or the patient's ability level after one or more treatments have been administered to the patient.

In view of the foregoing, potential treatments can be determined. Potential treatments can include treatments that could be administered to the patient to improve one or more activities to be performed by the patient. Each potential treatment can have an associated expected outcome measure indicating the likely improvement to the patient's ability level if the treatment was administered to the patient along with a confidence metric indicating a likelihood that the patient would achieve the expected improvement. The intermediate scores and the ML score 138 can be calculated using one or more machine learning models as trained and described herein, and then one or more treatments can be administered to the patient. The one or more treatments can include one or more the determined potential treatments. In several examples, the administered treatment includes the potential treatment corresponding to the greatest ML score 138. In a variety of examples, the administered treatment includes the potential treatment with the greatest likelihood of achieving the expected improvement.

### Technical Operating Environment and Exemplary Computing Device

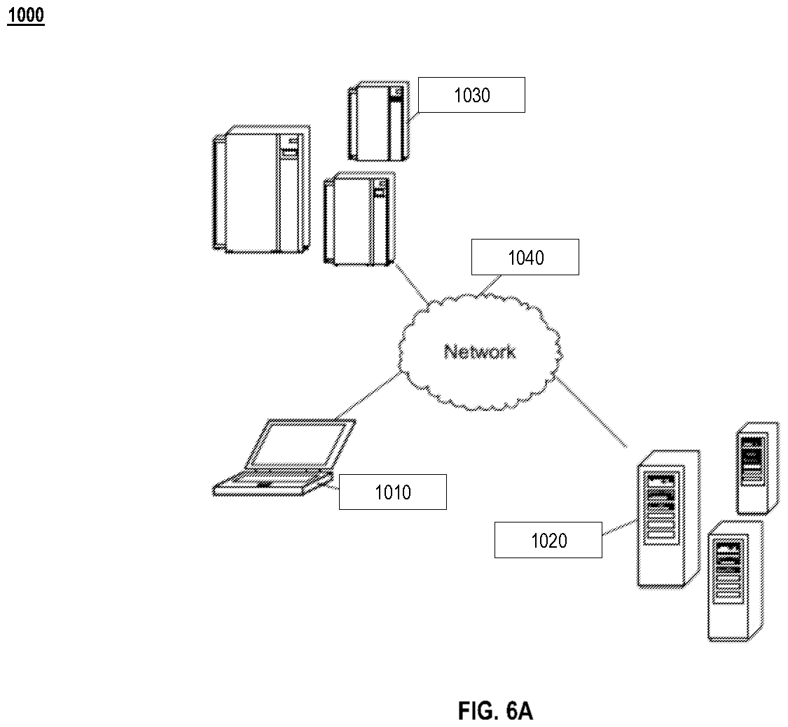

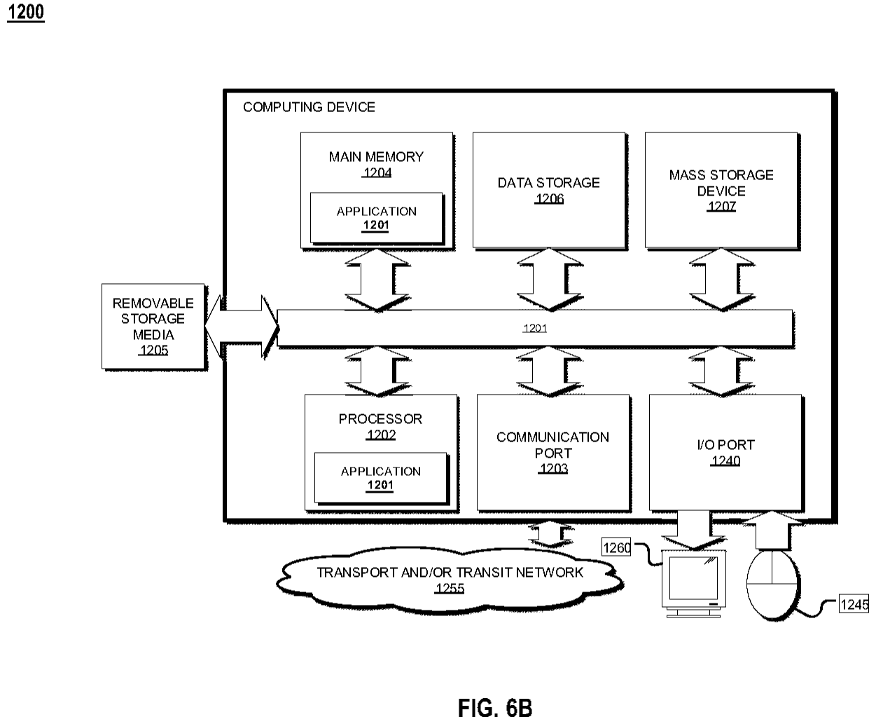

FIG. 6A shows an operating environment 1000. The operating environment 1000 can include at least one client device 1010, at least one database system 1020, and/or at least one server system 1030 in communication via a network 1040. It will be appreciated that the network connections shown are illustrative and any means of establishing a communications link between the computers can be used. The existence of any of various network protocols such as TCP/IP, Ethernet, FTP, HTTP and the like, and of various wireless communication technologies such as GSM, CDMA, WiFi, and LTE, is presumed, and the various computing devices described herein can be configured to communicate using any of these network protocols or technologies. Any of the devices and systems described herein can be implemented, in whole or in part, using one or more computing devices described with respect to FIG. 1 and FIG. 6B.

Client devices 1010 can obtain patient data and/or provide recommended treatment plans as described herein. Database systems 1020 can obtain, store, and provide a variety of patient data and/or treatment plans as described herein. Databases can include, but are not limited to relational databases, hierarchical databases, distributed databases, in-memory databases, flat file databases, XML databases, NoSQL databases, graph databases, and/or a combination thereof. Server systems 1030 can automatically generate scores from outcome measures using a variety of machine learning models trained or otherwise configured as described herein. The network 1040 can include a local area network (LAN), a wide area network (WAN), a wireless telecommunications network, and/or any other communication network or combination thereof.

The data transferred to and from various computing devices in the operating environment 1000 can include secure and sensitive data, such as confidential documents, customer personally identifiable information, and account data. Therefore, it can be desirable to protect transmissions of such data using secure network protocols and encryption, and/or to protect the integrity of the data when stored on the various computing devices. For example, a file-based integration scheme or a service-based integration scheme can be utilized for transmitting data between the various computing devices. Data can be transmitted using various network communication protocols. Secure data transmission protocols and/or encryption can be used in file transfers to protect the integrity of the data, for example, File Transfer Protocol (FTP), Secure File Transfer Protocol (SFTP), and/or Pretty Good Privacy (PGP) encryption. In many examples, one or more web services can be implemented within the various computing devices. Web services can be accessed by authorized external devices and users to support input, extraction, and manipulation of data between the various computing devices in the operating environment 1000. Web services built to support a personalized display system can be cross-domain and/or cross-platform, and can be built for enterprise use. Data can be transmitted using the Secure Sockets Layer (SSL) or Transport Layer Security (TLS) protocol to provide secure connections between the computing devices. Web services can be implemented using the WS-Security standard, providing for secure SOAP messages using XML encryption. Specialized hardware can be used to provide secure web services. For example, secure network appliances can include built-in features such as hardware-accelerated SSL and HTTPS, WS-Security, and/or firewalls. Such specialized hardware can be installed and configured in the operating environment 1000 in front of one or more computing devices such that any external devices can communicate directly with the specialized hardware.

Referring to FIG. 6B, a computing device 1200 is illustrated which may be included within the operating environment 1000 of FIG. 6A and be configured, via one or more of an application 1211 or computer-executable instructions, to execute functionality associated with quantifying patient improvement, as described herein. More particularly, in some examples, aspects of the methods herein may be translated to software or machine-level code, which may be installed to and/or executed by the computing device 1200 such that the computing device 1200 is configured for AI-driven patient improvement quantification, among other functionality described herein. It is contemplated that the computing device 1200 may include any number of devices, such as personal computers, server computers, hand-held or laptop devices, tablet devices, multiprocessor systems, microprocessor-based systems, set top boxes, programmable consumer electronic devices, network PCs, minicomputers, mainframe computers, digital signal processors, state machines, logic circuitries, distributed computing environments, and the like.

The computing device 1200 may include various hardware components, such as a processor 1202, a main memory 1204 (e.g., a system memory), and a system bus 1201 that couples various components of the computing device 1200 to the processor 1202. The system bus 1201 may be any of several types of bus structures including a memory bus or memory controller, a peripheral bus, and a local bus using any of a variety of bus architectures. For example, such architectures may include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus also known as Mezzanine bus.

The computing device 1200 may further include a variety of memory devices and computer-readable media 1207 that includes removable/non-removable media and volatile/nonvolatile media and/or tangible media, but excludes transitory propagated signals. Computer-readable media 1207 may also include computer storage media and communication media. Computer storage media includes removable/non-removable media and volatile/nonvolatile media implemented in any method or technology for storage of information, such as computer-readable instructions, data structures, program modules or other data, such as RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium that may be used to store the desired information/data and which may be accessed by the computing device 1200. Communication media includes computer-readable instructions, data structures, program modules, or other data in a modulated data signal such as a carrier wave or other transport mechanism and includes any information delivery media. The term “modulated data signal” means a signal that has one or more of its characteristics set or changed in such a manner as to encode information in the signal. For example, communication media may include wired media such as a wired network or direct-wired connection and wireless media such as acoustic, RF, infrared, and/or other wireless media, or some combination thereof. Computer-readable media may be embodied as a computer program product, such as software stored on computer storage media.

The main memory 1204 includes computer storage media in the form of volatile/nonvolatile memory such as read only memory (ROM) and random access memory (RAM). A basic input/output system (BIOS), containing the basic routines that help to transfer information between elements within the computing device 1200 (e.g., during start-up) is typically stored in ROM. RAM typically contains data and/or program modules that are immediately accessible to and/or presently being operated on by processor 1202. Further, data storage 1206 in the form of Read-Only Memory (ROM) or otherwise may store an operating system, application programs, and other program modules and program data.

The data storage 1206 may also include other removable/non-removable, volatile/nonvolatile computer storage media. For example, the data storage 1206 may be: a hard disk drive that reads from or writes to non-removable, nonvolatile magnetic media; a magnetic disk drive that reads from or writes to a removable, nonvolatile magnetic disk; a solid state drive; and/or an optical disk drive that reads from or writes to a removable, nonvolatile optical disk such as a CD-ROM or other optical media. Other removable/non-removable, volatile/nonvolatile computer storage media may include magnetic tape cassettes, flash memory cards, digital versatile disks, digital video tape, solid state RAM, solid state ROM, and the like. The drives and their associated computer storage media provide storage of computer-readable instructions, data structures, program modules, and other data for the computing device 1200.

A user may enter commands and information through a user interface 1240 (displayed via a monitor 1260) by engaging input devices 1245 such as a tablet, electronic digitizer, a microphone, keyboard, and/or pointing device, commonly referred to as mouse, trackball or touch pad. Other input devices 1245 may include a joystick, game pad, satellite dish, scanner, or the like. Additionally, voice inputs, gesture inputs (e.g., via hands or fingers), or other natural user input methods may also be used with the appropriate input devices, such as a microphone, camera, tablet, touch pad, glove, or other sensor. These and other input devices 1245 are in operative connection to the processor 1202 and may be coupled to the system bus 1201, but may be connected by other interface and bus structures, such as a parallel port, game port or a universal serial bus (USB). The monitor 1260 or other type of display device may also be connected to the system bus 1201. The monitor 1260 may also be integrated with a touch-screen panel or the like.

The computing device 1200 may be implemented in a networked or cloud-computing environment using logical connections of a network interface 1203 to one or more remote devices, such as a remote computer. The remote computer may be a personal computer, a server, a router, a network PC, a peer device or other common network node, and typically includes many or all of the elements described above relative to the computing device 1200. The logical connection may include one or more local area networks (LAN) and one or more wide area networks (WAN), but may also include other networks. Such networking environments are commonplace in offices, enterprise-wide computer networks, intranets and the Internet.

When used in a networked or cloud-computing environment, the computing device 1200 may be connected to a public and/or private network through the network interface 1203. In such examples, a modem or other means for establishing communications over the network is connected to the system bus 1201 via the network interface 1203 or other appropriate mechanism. A wireless networking component including an interface and antenna may be coupled through a suitable device such as an access point or peer computer to a network. In a networked environment, program modules depicted relative to the computing device 1200, or portions thereof, may be stored in the remote memory storage device.

Certain examples are described herein as including one or more modules. Such modules are hardware-implemented, and thus include at least one tangible unit capable of performing certain operations and may be configured or arranged in a certain manner. For example, a hardware-implemented module may comprise dedicated circuitry that is permanently configured (e.g., as a special-purpose processor, such as a field-programmable gate array (FPGA) or an application-specific integrated circuit (ASIC)) to perform certain operations. A hardware-implemented module may also comprise programmable circuitry (e.g., as encompassed within a general-purpose processor or other programmable processor) that is temporarily configured by software or firmware to perform certain operations. In some example examples, one or more computer systems (e.g., a standalone system, a client and/or server computer system, or a peer-to-peer computer system) or one or more processors may be configured by software (e.g., an application or application portion) as a hardware-implemented module that operates to perform certain operations as described herein.

Accordingly, the term “hardware-implemented module” encompasses a tangible entity, be that an entity that is physically constructed, permanently configured (e.g., hardwired), or temporarily configured (e.g., programmed) to operate in a certain manner and/or to perform certain operations described herein. Considering examples in which hardware-implemented modules are temporarily configured (e.g., programmed), each of the hardware-implemented modules need not be configured or instantiated at any one instance in time. For example, where the hardware-implemented modules comprise a general-purpose processor configured using software, the general-purpose processor may be configured as respective different hardware-implemented modules at different times. Software may accordingly configure the processor 1202, for example, to constitute a particular hardware-implemented module at one instance of time and to constitute a different hardware-implemented module at a different instance of time.

Hardware-implemented modules may provide information to, and/or receive information from, other hardware-implemented modules. Accordingly, the described hardware-implemented modules may be regarded as being communicatively coupled. Where multiple of such hardware-implemented modules exist contemporaneously, communications may be achieved through signal transmission (e.g., over appropriate circuits and buses) that connect the hardware-implemented modules. In examples in which multiple hardware-implemented modules are configured or instantiated at different times, communications between such hardware-implemented modules may be achieved, for example, through the storage and retrieval of information in memory structures to which the multiple hardware-implemented modules have access. For example, one hardware-implemented module may perform an operation, and may store the output of that operation in a memory device to which it is communicatively coupled. A further hardware-implemented module may then, at a later time, access the memory device to retrieve and process the stored output. Hardware-implemented modules may also initiate communications with input or output devices.

Computing systems or devices referenced herein may include desktop computers, laptops, tablets e-readers, personal digital assistants, smartphones, gaming devices, servers, and the like. The computing devices may access computer-readable media that include computer-readable storage media and data transmission media. In some examples, the computer-readable storage media are tangible storage devices that do not include a transitory propagating signal. Examples include memory such as primary memory, cache memory, and secondary memory (e.g., DVD) and other storage devices. The computer-readable storage media may have instructions recorded on them or may be encoded with computer-executable instructions or logic that implements aspects of the functionality described herein. The data transmission media may be used for transmitting data via transitory, propagating signals or carrier waves (e.g., electromagnetism) via a wired or wireless connection.

One or more aspects discussed herein can be embodied in computer-usable or readable data and/or computer-executable instructions, such as in one or more program modules, executed by one or more computers or other devices as described herein. Generally, program modules include routines, programs, objects, components, data structures, and the like, that perform particular tasks or implement particular abstract data types when executed by a processor in a computer or other device. The modules can be written in a source code programming language that is subsequently compiled for execution, or can be written in a scripting language such as (but not limited to) HTML or XML. The computer executable instructions can be stored on a computer readable medium such as a hard disk, optical disk, removable storage media, solid-state memory, RAM, and the like. As will be appreciated by one of skill in the art, the functionality of the program modules can be combined or distributed as desired in various examples. In addition, the functionality can be embodied in whole or in part in firmware or hardware equivalents such as integrated circuits, field programmable gate arrays (FPGA), and the like. Particular data structures can be used to more effectively implement one or more aspects discussed herein, and such data structures are contemplated within the scope of computer executable instructions and computer-usable data described herein. Various aspects discussed herein can be embodied as a method, a computing device, a system, and/or a computer program product.

Exemplary Software/Hardware Components

The machine learning architecture described herein may be implemented along with source files to gather and process data, train and test neural network as described, and then save and visualize the results. Exemplary hardware to execute functionality herein may include an AWS virtual machine (Ubuntu 18, 512 MB RAM, 1 core processor, 20 GB storage). Additional hardware may be implemented for computers that train the model or preprocess data. Code may be built in Python 3.6, and TensorFlow framework may be used for machine learning with Flask for a web application. Access can be provided to an API for those who desire to interact with the system 100 via a user interface or via POST requests. Other such features are contemplated.

Although the present invention has been described in certain specific aspects, many additional modifications and variations would be apparent to those skilled in the art. In particular, any of the various processes described above can be performed in alternative sequences and/or in parallel (on different computing devices) in order to achieve similar results in a manner that is more appropriate to the requirements of a specific application. It is therefore to be understood that the present invention can be practiced otherwise than specifically described without departing from the scope and spirit of the present invention. Thus, examples of the present invention should be considered in all respects as illustrative and not restrictive.

It should be understood from the foregoing that, while particular examples have been illustrated and described, various modifications can be made thereto without departing from the spirit and scope of the invention as will be apparent to those skilled in the art. Such changes and modifications are within the scope and teachings of this invention as defined in the claims appended hereto.

What is claimed is:

1. A method of quantifying rehabilitative progress via artificial intelligence, comprising:
accessing, by a computing device, a first dataset of input data for one or more outcome measures derived from a patient at a first point in time of rehabilitation;
accessing, by the computing device, a second dataset of the input data for the one or more outcome measures derived from the patient at a second point in time of the rehabilitation; and
generating, by the computing device applying the first dataset and the second dataset as inputs to a machine learning model, an output including a machine learning score that infers improvement of the patient from the first point in time to the second point in time, the machine learning model trained to map the inputs to the output to minimize a cost function defined by the machine learning model and maximize the dissimilarity between the patient between the first point in time and the second point in time.

2. The method of claim 1, further comprising:
generating, by the computing device executing the machine learning model in view of the inputs, a first intermediate score associated with the first dataset and a second intermediate score associated with the second dataset; and
computing a difference between the first intermediate score and the second intermediate score to derive the machine learning score.

3. The method of claim 1, further comprising generating by the computing device,
applying at least a portion of the input data to the machine learning model to generate a distribution of intermediate scores; the distribution of intermediate scores reflecting computed changes in each of the one or more outcome measures at respective points in time, greater scores of the distribution of intermediate scores reflecting greater improvement of the patient made during the rehabilitation.

4. The method of claim 1, wherein the machine learning model is trained to learn certain ones of the one or more outcome measures that represent a maximal dissimilarity of the patient from the first point in time to the second point in time.

5. The method of claim 2, wherein the machine learning model comprises a Siamese neural network that includes an input layer defining a node for each outcome measure of the one or more outcome measures, and an output layer that includes a node that provides intermediate scores including the first intermediate score and the second intermediate score.

6. The method of claim 1, wherein the cost function is defined as:
J min(s1,s2)=−mean(s2−s1)/std(s2−s1)
wherein S2 corresponds to the second point in time and S1 corresponds to the first point in time, and the cost function assists the machine learning model during training to maximize the difference between S2 and S1.

7. The method of claim 1, wherein the first dataset and the second dataset correspond to phases of rehabilitation of the patient, and the function is a contrastive objective function that uses an assumption of patient improvement from the first point in time to the second point in time.

8. The method of claim 1, further comprising:
normalizing, by the computing device, the first dataset and the second dataset by rescaling each outcome measure from the first dataset and the second dataset to a range [0,1] using the minimum and maximum values for each outcome measure.

9. The method of claim 1, further comprising:
determining, by the computing device, a suggested activity for the patient based on the outcome measure; and
transmitting, by the computing device, the suggested activity to an end user device.

10. The method of claim 1, wherein the one or more outcome measures includes any metric configured as a numeric value informative as to a change in the patient.

11. The method of claim 1, wherein during machine learning the computing device derives an equation defining a plurality of computations performed by the machine learning model when executed, parameters of the equation being trained using the cost function to find the largest difference in patients between two points in time.

12. The method of claim 11, wherein the machine learning model during training modifies the parameters to minimize the cost function based on training data defining outcome measures fed to the machine learning model during training.

13. The method of claim 12, further comprising feeding incrementally the machine learning model with additional outcome measures training data and updating the parameters.

14. The method of claim 1, further comprising:
for each feature in a feature matrix defined by the first dataset, appending an additional column to the first dataset to serve as a mask for identifying missing data; and
assigning a value of 1 to reflect population of a value for a given outcome measure.

15. The method of claim 14, further assigning a value of 0 to reflect missing data.
